(19) United States
(12) Patent Application Publication
(10) Pub. No.: US 2005/0021128 A1
(43) Pub. Date: Jan. 27, 2005

(54) Compliant, porous, rolled stent

(75) Inventors: James E. Nakahama, Santa Rosa, CA (US); Todd D. Campbell, Petaluma, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/891,556

(22) Filed: Jul. 15, 2004

Related U.S. Application Data
(60) Provisional application No. 60/489,682, filed on Jul. 24, 2003.

(51) Int. Cl. A61F 2/06

(52) U.S. Cl. 623/1.15

(57) ABSTRACT

The invention provides a compliant, porous, rolled stent, comprising a stent framework configured as a rhomboid having two short sides and two long sides. The stent framework includes a plurality of slits formed parallel to the short sides of the rhomboid, edge portions adjacent to the long sides of the rhomboid being unslit. The stent framework is rolled at an angle such that the long sides of the rhomboid overlap one another to form a tubular structure. The tubular structure has a spiral backbone formed by the unslit edge portions adjacent to the long sides of the rhomboid. The short sides of the rhomboid form the proximal and distal ends of the stent.

### RELATED APPLICATIONS

This application claims the benefit of U.S. Provisional Patent Application 60/489,682 filed Jul. 24, 2003.

### TECHNICAL FIELD

This invention relates generally to biomedical devices that are used for treating vascular conditions. More specifically, the invention relates to a compliant, porous, rolled stent.

### BACKGROUND OF THE INVENTION

Stents are generally cylindrical-shaped devices that are radially expandable to hold open a segment of a vessel or other anatomical lumen after implantation into the body lumen. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire lumen that is part of the delivery device. After the stent is positioned across the lesion, it is expanded by the delivery device, causing the diameter of the stent to expand. For a self-expanding stent, a sheath or other restraint is removed from the stent, allowing it to expand.

Stents are commonly used following, percutaneous transluminal coronary angioplasty (PTCA). During PTCA, a balloon catheter device is inflated within a stenotic blood vessel to dilate the vessel. The stenosis may be the result of a lesion such as a plaque or thrombus. When inflated, the pressurized balloon exerts a compressive force on the lesion, thereby increasing the inner diameter of the affected vessel and producing improved blood flow. Soon after the procedure, however, a significant proportion of treated vessels restenose.

To prevent restenosis, a stent, constructed of a metal or polymer, is implanted within the vessel to maintain lumen size. The stent acts as a scaffold to support the lumen in an open position. Configurations of stents include a cylindrical tube defined by a mesh, a coil, interconnected stents, or like segments. Exemplary balloon-expandable stents are disclosed in U.S. Pat. No. 4,739,762 to Palmaz, and U.S. Pat. No. 5,421,955 to Lau et al. Exemplary self-expanding stents are disclosed in U.S. Pat. No. 5,246,445 to Yachia et al., U.S. Pat. No. 5,824,053 to Khosravi et al., and U.S. Pat. No. 6,533,905 to Johnson et al.

Prior art stents have displayed a number of drawbacks. Conventional mesh and tubular stents may be too rigid to easily negotiate tortuous vessels and may straighten out the natural curves in a vessel when deployed. In addition, tubular stents such as that disclosed in U.S. Pat. No. 6,533,905 to Johnson et al. offer no openings for endothelial growth through the stent, which may result in restenosis at the ends of the stents. While mesh and helical wire stents permit endothelial growth, the minimal surface area of such stents may result in limited support for the wall of the vessel and may expose the bloodstream to plaque or other embolic material attached to the wall of the vessel. In addition, mesh and helical wire stents may offer little surface area for adhering drug coatings and thus are limited in their ability to deliver drugs to the wall of a vessel.

Helical wire stents such as that disclosed in U.S. Pat. No. 5,246,445 to Yachia et al. present additional disadvantages. The free ends of these stents may flare out when delivered, injuring the wall of the vessel, or may protrude into the blood flow, which is thought to promote thrombosis. Because helical stents are generally wound tightly for delivery, the free ends may also whip around the catheter at high speed as they unwind, again injuring the wall of a vessel or possibly dislodging pieces of plaque that may result in embolization. Helical stents may also experience considerable longitudinal shortening after they are fully unwound, possibly resulting in improper placement of the stent. Localized slipping or migration of individual turns of a coil of a helical stent may also result in placement problems.

One attempt at addressing some of these problems is disclosed in U.S. Pat. No. 5,824,053 to Khosravi et al., which describes a helical mesh coil with a band width equal to at least one-quarter to one-third of the maximum expanded circumference of the stent. The helical mesh has openings forming a lattice that provides about 60% or more open space. The relatively small band width is intended to limit the amount of foreshortening and the speed at which the device uncoils when deployed. The lattice is intended to provide openings through which endothelialization may take place. While this device addresses some of the problems described above, it does not entirely eliminate the disadvantages resulting from helical stents with free ends. The free ends of the stent may still flare out when balloon expanded, while the minimal number of windings may limit the flexibility and compliance of the stent. In addition, the turns of the stent are not linked or stabilized, allowing individual turns to slip or migrate and possibly allowing the stent to stretch, reducing its diameter.

Therefore, it would be desirable to provide a stent that overcomes the aforementioned and other disadvantages.

### SUMMARY OF THE INVENTION

One aspect of the present invention is a compliant, porous, rolled stent, comprising a stent framework configured as a rhomboid having two short sides and two long sides. The stent framework includes a plurality of slits formed parallel to the short sides of the rhomboid, edge portions adjacent to the long sides of the rhomboid being unslit. The stent framework is rolled at an angle such that the long sides of the rhomboid overlap one another to form a tubular structure. The tubular structure has a spiral backbone formed by the unslit edge portions adjacent to the long sides of the rhomboid. The short sides of the rhomboid form the proximal and distal ends of the stent.

Another aspect of the present invention is a system for treating a vascular condition, comprising a catheter and a stent releasably coupled to the catheter. The stent includes a stent framework configured as a rhomboid having two short sides and two long sides. The stent framework includes a plurality of slits formed parallel to the short sides of the rhomboid, edge portions adjacent to the long sides of the rhomboid being unslit. The stent framework is rolled at an angle such that the long sides of the rhomboid overlap one another to form a tubular structure. The tubular structure has a spiral backbone formed by the unslit edge portions adjacent to the long sides of the rhomboid. The short sides of the rhomboid form the proximal and distal ends of the stent.

A further aspect of the present invention is a method of making a system for treating a vascular condition. A flat sheet of material is formed into a rhomboid having two long sides and two short sides. A plurality of slits are formed into the rhomboid, the slits being parallel to the short sides of the rhomboid, an edge portion adjacent to each side of the rhomboid being unslit. The rhomboid is rolled such that the long sides overlap one another to form a tubular stent having a spiral backbone, the spiral backbone being formed by the unslit edge portions adjacent to the long sides of the rhomboid. The short sides of the rhomboid form the proximal and distal ends of the stent. A catheter is provided. The stent is releasably coupled to the catheter.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1A is an illustration of one embodiment of a stent in accordance with the present invention;

FIG. 1B is an illustration of the stent of FIG. 1A, showing the stent reduced in size and in a preliminary, unrolled configuration;

FIG. 2 is an illustration of one embodiment of a system for treating a vascular condition, in accordance with the present invention;

FIG. 3 is a flow diagram of one embodiment of a method of making a system for treating a vascular condition, in accordance with the present invention.

### DETAILED DESCRIPTION OF THE PRESENTLY PREFERRED EMBODIMENTS

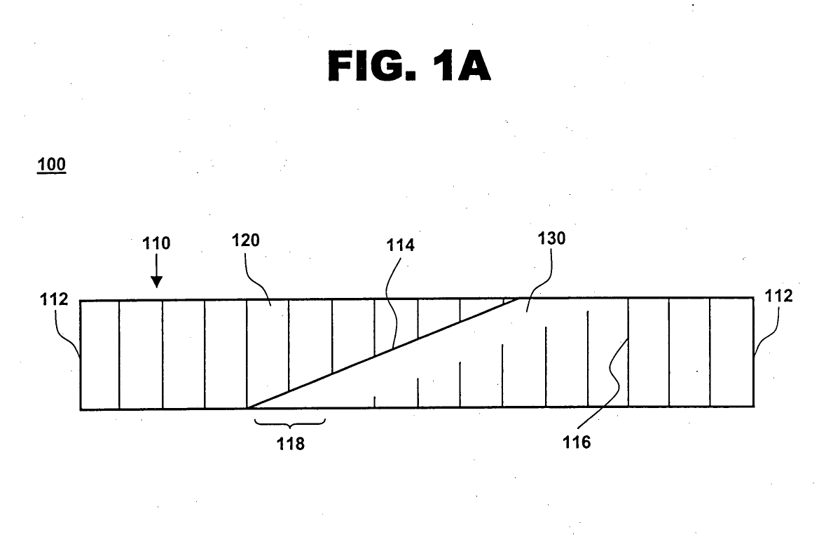

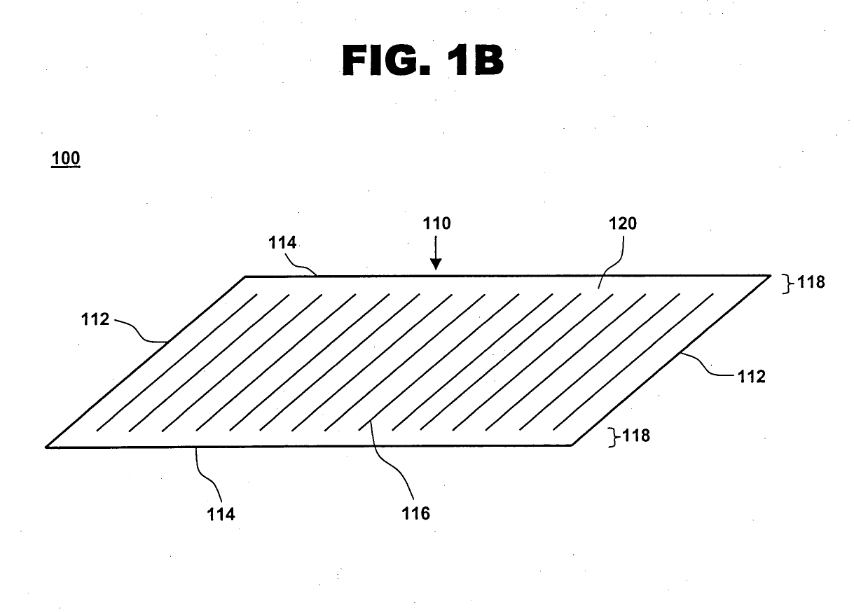

One aspect of the present invention is a compliant, porous, rolled stent. One embodiment of the stent, in accordance with the present invention, is illustrated in FIGS. 1A and 1B at 100. A completed stent is shown in FIG. 1A, while the same stent is shown reduced in size and in a preliminary, unrolled configuration in FIG. 1B. Stent 100 includes a stent framework 110 and a therapeutic coating 120. Stent framework 110 has two short sides 112 and two long sides 114 and includes a plurality of slits 116 formed parallel to short sides 112. Edge portions 118 adjacent to long sides 114 are unslit and form a spiral backbone 130 in the rolled stent. Short sides 112 form the proximal and distal ends of stent 100.

Stent framework 110 may be made of a wide variety of medical implantable materials, such as a shape-memory material, a biocompatible material, a biodegradable material, a metal, a ceramic, a polymer, and combinations thereof. For example, the framework may comprise a shape-memory material such as a nickel-titanium or nickel-titanium-copper alloy or a biodegradable polymer such as polylactide (PLA).

Stent framework 110 is configured as a rhomboid. As seen best in FIG. 1B, the rhomboid has two short sides 112 and two long sides 114. In the present embodiment, the long sides of the rhomboid are approximately twice as long as the short sides, the long sides being, for example, 20 millimeters in length, while the short sides are 10 millimeters in length. Interior angles of the rhomboid may be, for example, two 40-degree angles and two 140-degree angles. Stent framework 110 may be formed from a flat sheet having a thickness in the range of 10 to 50 microns, with a preferred thickness of approximately 25 microns.

Stent framework 110 includes a plurality of slits 116. The slits are formed parallel to the short sides 112 of the rhomboid and extend toward but not through the long sides 114 of the rhomboid, leaving edge portions 118 of the rhomboid adjacent to the long sides unslit. The number of slits formed into the stent framework may vary, with more slits typically producing a more compliant stent. A more compliant stent is a stent with more capability to bend during delivery of the stent to a target location within a vessel and more capability to support the vessel without simultaneously straightening the vessel upon deployment. The number of slits also determines the porosity of the finished stent. In an alternate embodiment, a slot may be used in place of a slit. Use of a slot over a slit may be beneficial in certain applications, but the frictional engagement inherent in the use of a slit allows for greater resistance to deformation and control of the expansion. The stent as shown in FIG. 1 comprises a slit, but those of ordinary skill in the art will readily recognize that a slot could be employed in place of the slit.

While stent 100 includes therapeutic coating 120, a stent in accordance with the present invention may be either coated or uncoated. Therapeutic coating 120 may include a therapeutic agent such as an antineoplastic agent, an antiproliferative agent, an antibiotic, an antithrombogenic agent, an anticoagulant, an antiplatelet agent, an anti-inflammatory agent, combinations of the above, and the like. The coating may comprise a material including, but not limited to, a biodurable polycarbonate-based aromatic or aliphatic urethane, other urethanes or polyurethanes, polylactide (PLA), poly-l-lactic acid (PLLA), polyglycolic acid (PGA) polymer, poly (e-caprolactone) (PCL), polyacrylates, polymethacrylates, polycaprolactone (PCL), polymethylmethacrylate (PMMA), combinations and/or copolymers of the above, and the like.

The stent framework is rolled at an angle such that long sides 114 overlap one another to form a tubular structure. When stent 100 is rolled correctly, unslit edge portions 118 spiral around the stent, forming a spiral backbone 130. This backbone allows the stent to bend freely in lateral directions, while stabilizing the stent longitudinally, thereby preventing substantial shortening or lengthening of the stent during and following deployment of the stent. A stent having the dimensions described above, i.e., 20-millimeter long sides and 10-millimeter short sides, will have a rolled length of approximately 14 millimeters. The angle where the long side and the short sides abut is an angle alpha. Angle alpha has a complementary angle beta. In one embodiment, alpha is an angle between approximately 30 and approximately 60 degrees, and beta is the complementary angle computed with the formula 180−alpha. In another embodiment, beta is an angle between approximately 100 and approximately 120 degrees, and alpha is computed with the formula 180-beta. In yet another embodiment, alpha is between approximately 10 and approximately 30 degrees and beta is the complementary angle computed with the formula 180−alpha. In yet another embodiment, alpha is an angle between approximately 60 degrees and approximately 80 degrees and beta is the complementary angle computed with the formula 180−alpha. Those of ordinary skill in the art will readily recognize that the denomination of alpha and beta is obvious, with alpha being an angle less than 90 degrees, and beta being an angle greater than 90 degrees such that alpha+beta=180 degrees. Short sides 112 form the proximal and distal ends of the stent.

The slits extend across a length of stent, as shown in FIG. 1B. In one embodiment, the slits extend across approximately 75% of the width of the stent. In another embodiment, the slits extend across approximately 30% to 90% of the width of the stent. In another embodiment, the slits extend across a substantial width of the stent. The slits may extend to within approximately 5% of the width of the stent. For example, for a stent with 20-millimeter long sides and 10-millimeter short sides, the slits may extend to within between approximately 1 and approximately 5 millimeters of the edge of the stent. In another example, and with a similarly dimensioned stent, the slits extend between approximately 5 and approximately 8 millimeters from the edge of the stent.

Stent 100 may be circumferentially compressed to form a contracted state for delivery within a vessel and may substantially return to an expanded state when deployed within the vessel. Stent 100 may undergo little or no longitudinal shortening between the contracted state and the expanded state as a result of the stent coiling upon itself and thereby maintaining a largely constant length.

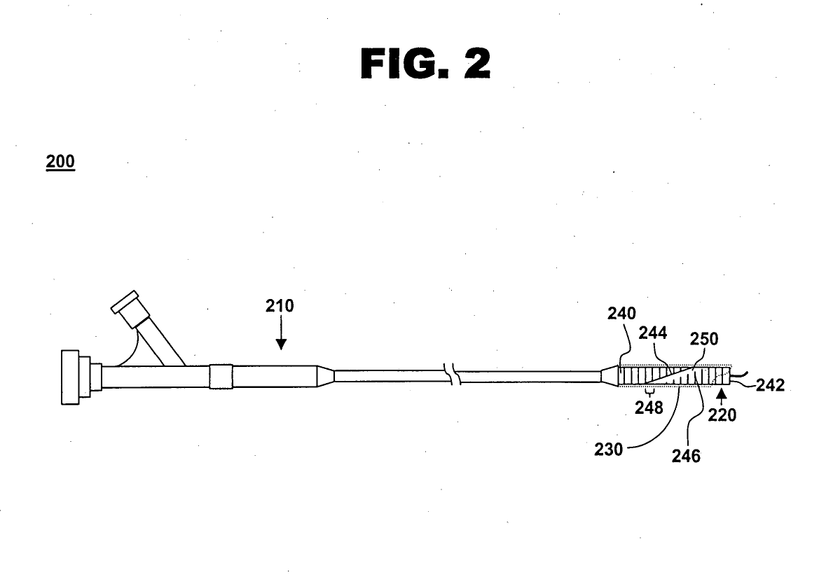

Another aspect of the present invention is a system for treating a vascular condition. One embodiment of the system, in accordance with the present invention, is illustrated in FIG. 2 at 200. System 100 comprises a catheter 210 and a stent 220. Catheter 210 includes a sheath 230. Stent 220 includes a stent framework 240 having two short sides 242 and two long sides 244. Stent framework 240 includes a plurality of slits 246 formed parallel to short sides 242. Edge portions 248 adjacent to long sides 244 are unslit and form a spiral backbone 250 in the rolled stent. Short sides 242 form the proximal and distal ends of stent 220. System 200 may include a therapeutic coating (not shown) disposed on at least a portion of stent 220.

Catheter 210 may be any catheter known in the art that is appropriate for delivering a stent to a treatment site within a vessel. In this embodiment, catheter 210 includes a sheath 230 that retracts to allow expansion of stent 220. Depending on the material or materials comprising the stent, catheter 210 may, alternatively, include at least two retaining members positioned adjacent to the distal and proximal ends of the stent that retract to allow expansion of a self-expanding stent. Where the stent is not self-expanding, catheter 210 may include a balloon used to expand the stent. Combinations of the above may be desirable, for example a balloon may be included to assist the expansion of a self-expanding stent that is retained by a sheath or retaining members prior to deployment.

Stent 220 is releasably coupled to catheter 210. In the present embodiment, stent 220 includes a stent framework 240 comprising a shape-memory material such as a nickel-titanium or nickel-titanium-copper alloy. Stent framework 240 may, alternatively, be made of a wide variety of medical implantable materials including, but not limited to, a biocompatible material, a biodegradable material, a metal, a polymer, and combinations thereof.

Stent framework 240 is configured as a rhomboid having two short sides 242 and two long sides 244 and includes a plurality of slits 246. The slits are formed parallel to the short sides 242 of the rhomboid and extend toward but not through the long sides 244 of the rhomboid, leaving edge portions 248 of the rhomboid adjacent to the long sides unslit. The number of slits formed into the stent framework may vary, with more slits typically producing a more compliant stent, that is a stent with more capability to bend during delivery of the stent to a target location within a vessel and more capability to support the vessel without simultaneously straightening the vessel upon deployment. The number of slits also determines the porosity of the finished stent.

A therapeutic coating (not shown) may be disposed on at least a portion of stent 220. The therapeutic coating may include a therapeutic agent such as an antineoplastic agent, an antiproliferative agent, an antibiotic, an antithrombogenic agent, an anticoagulant, an antiplatelet agent, an anti-inflammatory agent, combinations of the above, and the like. The coating may comprise a material including, but not limited to, a biodurable polycarbonate-based aromatic or aliphatic urethane, other urethanes or polyurethanes, polylactide (PLA), poly-l-lactic acid (PLLA), polyglycolic acid (PGA) polymer, poly (e-caprolactone) (PCL), polyacrylates, polymethacrylates, polycaprolactone (PCL), polymethylmethacrylate (PMMA), combinations and/or copolymers of the above, and the like. Combinations of polymers with therapeutic agents may also be used in the coating.

The stent framework is rolled at an angle such that long sides 244 overlap one another to form a tubular structure. When stent 220 is rolled correctly, unslit edge portions 248 spiral around the stent, forming a spiral backbone 250. This backbone allows the stent to bend freely in lateral directions, while also stabilizing the stent longitudinally, thereby preventing substantial shortening or lengthening of the stent during and following deployment of the stent. Short sides 242 form the proximal and distal ends of the stent.

Stent 220 may be circumferentially compressed to form a contracted state for delivery within a vessel and may substantially return to an expanded state when deployed within the vessel. Stent 220 may undergo little or no longitudinal shortening between the contracted state and the expanded state as a result of the stent coiling upon itself and thereby maintaining a largely constant length.

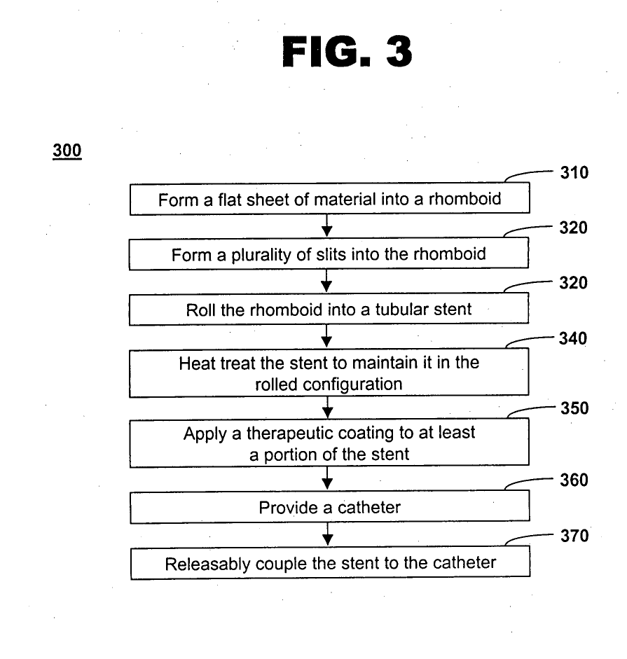

A further aspect of the present invention is a method of making a system for treating a vascular condition. FIG. 3 shows a flow diagram of one embodiment in accordance with the present invention at 300.

A flat sheet of material is formed into a rhomboid having two long sides and two short sides (Block 310). The rhomboid may be formed by, for example, laser cutting a rhomboidal shape into a flat sheet comprising a shape-memory material such as a nickel-titanium-copper alloy. The flat sheet may have a thickness in the range of 10 to 50 microns, with a preferred thickness of approximately 25 microns.

A plurality of slits is formed into the rhomboid (Block 320). The slits are formed parallel to the short sides of the rhomboid and extend toward but not through the long sides of the rhomboid, edge portions adjacent to the long sides of the rhomboid being unslit. The slits may be formed by, for example, laser or die cutting.

The rhomboid is rolled such that the long sides of the rhomboid overlap one another to form a tubular stent having a spiral backbone formed by the unslit edge portions adjacent to the long sides of the rhomboid, the short sides of the rhomboid forming the proximal and distal ends of the stent (Block 330). The short sides of the rhomboid are disposed to be parallel and orthogonal to the longitudinal axis of the stent when rolled. For example, this disposition is illustrated by numeral 112 as seen in FIG. 1A. This may be accomplished by, for example, rolling the rhomboid at an angle around a mandrel.

The stent may then be heat treated to maintain it in the rolled configuration (Block 340). For a shape-memory material such as a nickel-titanium-copper alloy, this comprises transitioning the material to an austenitic state by, for example, annealing the rolled stent in a salt pot. Where the stent comprises a material other than a shape-memory material, this step may be eliminated or a different method may be employed to maintain the stent in a rolled configuration.

A therapeutic coating may be applied to at least a portion of the stent (Block 350). The coating may be applied by a method such as infusing, dipping, spraying, pad printing, inkjet printing, rolling, painting, micro-spraying, wiping, electrostatic deposition, vapor deposition, epitaxial growth, and combinations thereof. Depending on the material or materials comprising the stent and the steps necessary to maintain the stent in a rolled configuration, the therapeutic coating may be applied either before or after rolling the stent.

A catheter is provided (Block 360). The catheter may be any catheter known in the art that is appropriate for delivering a stent to a lesion site identified for treatment. The stent is releasably coupled to the catheter (Block 360). Coupling the stent to the catheter involves circumferentially compressing the stent to form a contracted state for delivery within a vessel and retaining the stent to the catheter. When using a shape-memory material, a sheath or retaining members such as removable sutures or rings may be used to maintain the stent in the contracted state and retain the stent to the catheter. Where the stent comprises a material that is not self-expanding, the stent may simply be crimped onto an elastomeric balloon attached to the catheter.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

1. A compliant, porous, rolled stent, comprising:
a stent framework configured as a rhomboid having two short sides and two long sides, the stent framework including a plurality of slits formed parallel to the short sides of the rhomboid, an edge portion adjacent to each long side of the rhomboid being unslit, the stent framework being rolled at an angle such that the long sides of the rhomboid overlap one another to form a tubular structure having a spiral backbone formed by the unslit edge portions adjacent to the long sides of the rhomboid, the short sides of the rhomboid forming a proximal and a distal end of the stent.

2. The stent of claim 1 wherein the stent is circumferentially compressed to form a contracted state for delivery within a vessel and substantially returns to an expanded state when deployed within the vessel.

3. The stent of claim 2 wherein the stent undergoes little or no longitudinal shortening between the contracted state and the expanded state.

4. The stent of claim 1 wherein the stent framework is formed from a flat sheet having a thickness in the range of 10 to 50 microns.

5. The stent of claim 1 wherein the stent framework comprises a medical implantable material selected from a group consisting of a shape-memory material, a biocompatible material, a biodegradable material, a metal, a ceramic, a polymer, and combinations thereof.

6. The stent of claim 1 wherein the shape-memory material comprises a nickel-titanium alloy.

7. The stent of claim 1 wherein the shape-memory material comprises a nickel-titanium-copper alloy.

8. The stent of claim 1 further comprising:
a therapeutic coating disposed on at least a portion of the stent framework.

9. The stent of claim 8 wherein the therapeutic coating includes a therapeutic agent selected from a group consisting of an antineoplastic agent, an antiproliferative agent, an antibiotic, an antithrombogenic agent, an anticoagulant, an antiplatelet agent, and an anti-inflammatory agent.

10. A system for treating a vascular condition, comprising:
a catheter; and
a stent releasably coupled to the catheter, the stent including a stent framework configured as a rhomboid having two short sides and two long sides, the stent framework including a plurality of slits formed parallel to the short sides of the rhomboid, an edge portion adjacent to each long side of the rhomboid being unslit, the stent framework being rolled at an angle such that the long sides of the rhomboid overlap one another to form a tubular structure having a spiral backbone formed by the unslit edge portions adjacent to the long sides of the rhomboid, the short sides of the rhomboid forming a proximal and a distal end of the stent.

11. The system of claim 10 wherein the stent is circumferentially compressed to form a contracted state when coupled to the catheter and wherein the stent substantially returns to an expanded state when released from the catheter.

12. The system of claim 11 wherein the stent undergoes little or no longitudinal shortening between the contracted state and the expanded state.

13. The system of claim 10 wherein the catheter includes a balloon used to expand the stent.

14. The system of claim 10 wherein the catheter includes a sheath that retracts to allow expansion of the stent.

15. The system of claim 10 wherein the catheter includes at least two retaining members positioned adjacent to a distal and a proximal end of the stent that retract to allow expansion of the stent.

16. The system of claim 10 wherein the stent framework comprises a medical implantable material selected from a group consisting of a shape-memory material, a biocompatible material, a biodegradable material, a metal, a ceramic, a polymer, and combinations thereof.

17. The system of claim 16 wherein the shape memory material comprises a nickel-titanium alloy.

18. The system of claim 16 wherein the shape memory material comprises a nickel-titanium-copper alloy.

19. The system of claim 10 further comprising:
a therapeutic coating disposed on at least a portion of the stent.

20. The system of claim 19 wherein the therapeutic coating includes a therapeutic agent selected from a group consisting of an antineoplastic agent, an antiproliferative agent, an antibiotic, an antithrombogenic agent, an anticoagulant, an antiplatelet agent, and an anti-inflammatory agent.

21. A method of manufacturing a system for treating a vascular condition, comprising:
forming a flat sheet of material into a rhomboid having two short sides and two long sides;
forming a plurality of slits into the rhomboid, the slits being parallel to the short sides of the rhomboid, an edge portion adjacent to each long side of the rhomboid being unslit;
rolling the rhomboid such that the long sides overlap one another to form a tubular stent having a spiral backbone formed by the unslit edge portions adjacent to the long sides of the rhomboid, the short sides of the rhomboid forming a proximal and a distal end of the stent;
a catheter is provided; and
the stent is releasably coupled to the catheter.

22. The method of claim 21 further comprising:
heat treating the stent to maintain it in a rolled configuration.

23. The method of claim 21 further comprising:
applying a therapeutic coating to at least a portion of the stent.

24. The method of claim 22 wherein the therapeutic coating is applied by a method selected from the group consisting of infusing, dipping, spraying, pad printing, inkjet printing, rolling, painting, micro-spraying, wiping, electrostatic deposition, vapor deposition, epitaxial growth, and combinations thereof.
